(19) United States
(12) Patent Application Publication
(10) Pub. No.: US 2005/0283166 A1
(43) Pub. Date: Dec. 22, 2005

(54) Expandible snare

(75) Inventor: E. Skott Greenhalgh, Wyndmoor, PA (US)

Correspondence Address: SYNNESTVEDT & LECHNER, LLP, 2600 ARAMARK TOWER, 1101 MARKET STREET, PHILADELPHIA, PA, 191072950, US

(73) Assignee: Secant Medical, LLC, Perkasie, PA (US)

(21) Appl. No.: 11/151,979

(22) Filed: Jun. 14, 2005

Related U.S. Application Data
(60) Provisional application No. 60/580,666, filed on Jun. 17, 2004.

(51) Int. Cl. A61B 17/24

(52) U.S. Cl. 606/113

(57) ABSTRACT

A snare for capturing and removing emboli from vascular vessels is disclosed. The snare is formed from a flexible, resilient sheet attached to an elongated flexible tube and rod assembly. The rod fits within a bore of the tube and the tube and rod are movable relatively to one another. The sheet forms a generally conical basket, one part of which is attached to the tube, the other part being attached to the rod. Relative motion between the tube and the rod causes the basket to deform between a contracted state, wherein it may pass through the vascular vessel, to an expanded state, wherein it may receive and capture an embolus for removal from the vessel.

### FIELD OF THE INVENTION

This invention relates to snares for the removal of embolisms from vascular vessels in the treatment of strokes.

### BACKGROUND OF THE INVENTION

An ischemic stroke results when an artery carrying blood to a portion of the brain becomes blocked by an embolus. The embolus may be a blood clot or a fatty deposit which has broken free and is transported by the blood stream through the vascular system until it lodges in an artery within the brain that is too small to allow it to pass. The embolism or blockage of the artery reduces or totally halts the flow of blood to that portion of the brain normally fed by the now blocked artery, often with catastrophic consequences.

Each year, over 600,000 people in the United States suffer strokes and 27% of them die as a result. Only 10% of stroke victims achieve a full recovery, and 40% have moderate to severe impairments such as blindness, paralysis of the limbs, loss of speech function and loss of cognitive functions resulting from the death of oxygen-starved brain tissue.

It is preferred to take preventive measures against the occurrence of strokes. If detected early enough, a stroke may be treated with thrombolytic drugs which break up clots and operate to restore blood flow to the brain. Such treatment is not without increased risk of bleeding, however, which can cause additional brain damage. If the stroke victim arrives at a hospital too late for thrombolytic treatment (as most do), it is still advantageous to remove the blockage even though it will not restore the lost cerebral function or the dead tissue. Removal of the blockage will lessen the likelihood of additional strokes and prevent secondary effects, such as the release of excitotoxins by damaged neurons, cerebral edema as well as alterations in blood flow around the affected region, all of which contribute to additional neuronal death.

There is clearly a need for a minimally invasive device and technique for treating arterial embolisms by removing the emboli that cause strokes. Such a device will mitigate the risk of further strokes and further injury without itself presenting an increased risk of brain damage.

### SUMMARY OF THE INVENTION

The invention concerns a snare for capturing and removing an embolus from a vessel. The snare comprises an elongated flexible tube having a bore therethrough. An elongated flexible rod is positioned within the bore of the tube. An end of the rod projects outwardly from the tube. The rod and the tube are movable relatively to one another. A flexible sheet has a first portion attached to the rod and a second portion positioned in spaced apart relation to the first portion. The second portion is attached to the tube. The sheet is deformable between a contracted state, wherein the sheet is substantially positioned in proximity to the tube, and an expanded state, wherein the sheet extends outwardly from the tube to form a basket. The basket has an opening for receiving the embolus. Relative motion between the rod and the tube deforms the sheet between the expanded and contracted states.

Preferably, the flexible sheet comprises an elongated strip having one end attached to the end of the rod and an opposite and attached to the tube, the strip being helically wrapped around the rod and the tube. The strip is preferably formed of interlaced filamentary members.

In one embodiment, the strip has lengthwise extending edges oppositely disposed from one another. The strip is biased so as to bring the edges substantially into contact with one another forming a substantially closed surface defining the basket when the strip is in the expanded state.

The snare may also include a second flexible sheet attached to the rod and positioned in spaced relation to the first flexible sheet. The second flexible sheet is deformable outwardly from the rod to form a basket having an opening for receiving the embolus. A link element extends between the first and the second sheets. The link element connects the sheets to one another so that the second sheet is deformed into the expanded state in response to motion of the first sheet being deformed into the expanded state.

In another embodiment of a snare according to the invention, the snare comprises an elongated flexible tube having a bore therethrough. An elongated flexible rod is positioned within the bore. An end of the rod projects outwardly from the tube. The rod and the tube are slidably movable relatively to one another. A flexible sheet forms a basket for receiving the embolus. One end of the basket is attached to the rod, the other end forming an opening for receiving the embolus. The basket is biased into a contracted state wherein the sheet is positioned substantially adjacent to the rod. The basket is deformable from the contracted state to an expanded state wherein the sheet extends outwardly from the rod to form the basket. Relative sliding motion between the rod and the tube causes the tube to engage an inner surface of the basket thereby deforming the sheet outwardly into the expanded state.

In yet another embodiment, the snare comprises an elongated flexible tube having a bore therethrough. An elongated flexible rod is positioned within the bore of the tube. An end of the rod projects outwardly from the tube. The rod and the tube are slidably movable relatively to one another. A cap is positioned on the end of the rod. The cap defines a socket with an opening facing the tube. The socket is sized to receive an end of the tube upon relative sliding motion between the rod and the tube. A flexible sheet forms a basket having one end attached to the tube and an opposite end forming an opening for receiving the embolus. The basket is resiliently biased into an expanded state wherein the sheet extends outwardly from the tube. The basket is deformable from the expanded state into a contracted state wherein the sheet is positioned substantially adjacent to the tube upon relative sliding motion between the rod and the tube inserting the end of the tube within the socket of the cap. The cap engages and deforms the basket into the contracted state.

In yet another embodiment of a snare according to the invention, the snare comprises an elongated flexible rod. A flexible sheet forms a basket having one end attached to the rod, the opposite end forming an opening for receiving the embolus. The basket is biased into an expanded state where it extends outwardly from the rod. The sheet is deformable into a contracted state wherein the sheet is positioned substantially adjacent to the rod upon insertion of the rod into the vessel. The basket resiliently assumes the expanded state upon motion of the rod in a direction removing the basket from the vessel.

In another embodiment, the snare comprises an elongated flexible rod. A flexible sheet forms a basket for receiving the embolus. One end of the basket is attached to the rod, the other end forms an opening. The basket is biased into a contracted state wherein the sheet is positioned substantially adjacent to the rod. A balloon is mounted on the rod within the basket. The balloon is inflatable to deform the basket from the contracted state to an expanded state wherein the basket extends outwardly from the rod. The basket resiliently assumes the contracted state upon deflation of the balloon.

### BRIEF DESCRIPTION OF THE DRAWINGS

FIG. 1 is a partial longitudinal sectional view of a snare according to the invention shown in an expanded state;

FIG. 2 is a partial longitudinal sectional view of the snare shown in FIG. 1, but in a contracted state;

FIG. 3 is a side view of another embodiment of a snare shown in an expanded state;

FIG. 4 is a side view of the snare shown in FIG. 3, but in a contracted state;

FIGS. 5-8 are sectional views which illustrate use of the snare in a procedure for removal of an embolus from an artery;

FIG. 9 is a side view of another embodiment of a snare according to the invention shown in an expanded state;

FIG. 10 is a side view of the snare shown in FIG. 9 but in a contracted state;

FIG. 11 is a side view of another embodiment of a snare according to the invention;

FIG. 12 is a side view of another embodiment of a snare shown in a contracted state;

FIG. 13 is a side view of the snare shown in FIG. 12, but in an expanded state;

FIG. 14 is a plan view of a component of the snare shown in FIG. 12;

FIG. 15 is an end view of the snare shown in FIG. 12 in an expanded state;

FIG. 16 is a partial cut away view of another embodiment of a snare shown in a contracted state;

FIG. 17 is a partial cut away view of the snare shown in FIG. 16, but in an expanded state;

FIG. 18 is a partial cut away view of another embodiment of a snare shown in a contracted state;

FIG. 19 is a partial cut away view of the snare shown in FIG. 18, but in an expanded state;

FIG. 20 is a partial sectional view of another embodiment of a snare shown in an expanded state; and

FIG. 21 is a side view of the snare shown in FIG. 20 but in a contracted state.

### DETAILED DESCRIPTION OF THE EMBODIMENTS

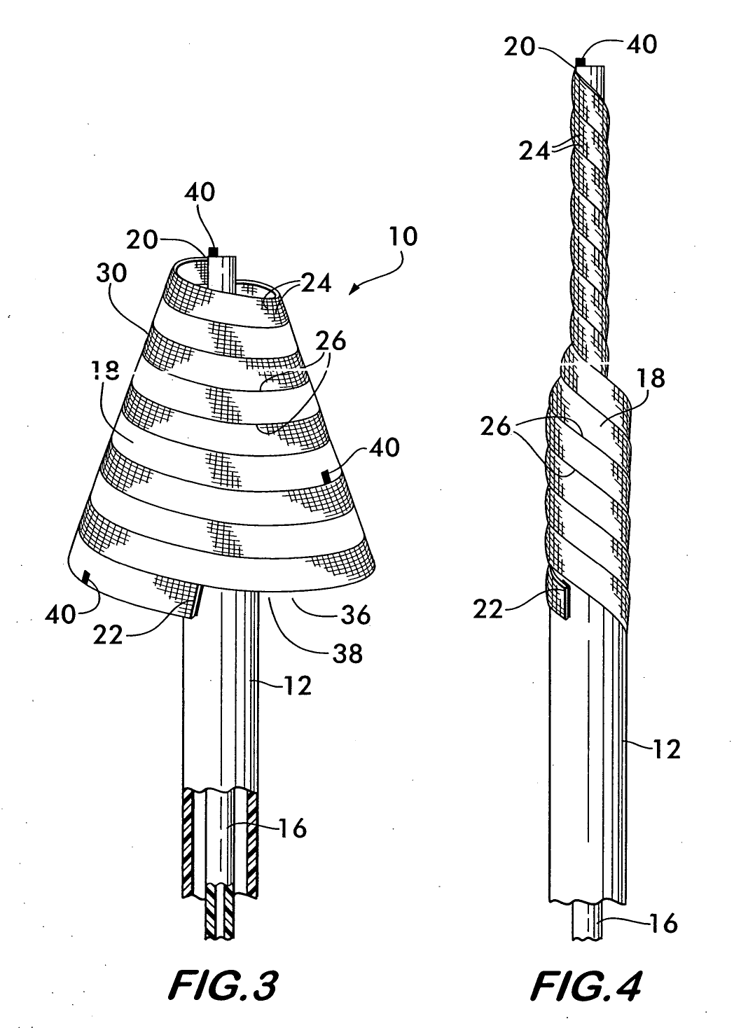

FIG. 1 shows an expandable snare 10 according to the invention. Snare 10 includes an elongated, flexible tube 12 with a bore 14 therethrough. An elongated, flexible rod 16 is positioned within bore 14. Tube 12 and rod 16 may be formed of nylon, polytetrafluoroethylene, polyester as well as resilient metals such as nitinol. Tube 12 may also be braid reinforced using high strength filaments such as stainless steel and eligiloy to prevent kinking. Rod 16 is movable relatively to tube 12 and may be twisted about its longitudinal axis and slid lengthwise within the bore 14. A flexible sheet, in this embodiment having the form of an elongated strip 18 has a first end 20 attached to the rod 16 and a second end 22 attached to tube 12. Strip 18 is preferably formed of interlaced filamentary members 24 and is resiliently biased into a helical shape surrounding the rod 16 and tube 12. Strip 18 has lengthwise extending edges 26 which may be in spaced apart relation to form a substantially open helix 28 as shown in FIG. 1, or the edges may be in abutting relation and form a substantially closed surface 30 as shown in FIG. 3.

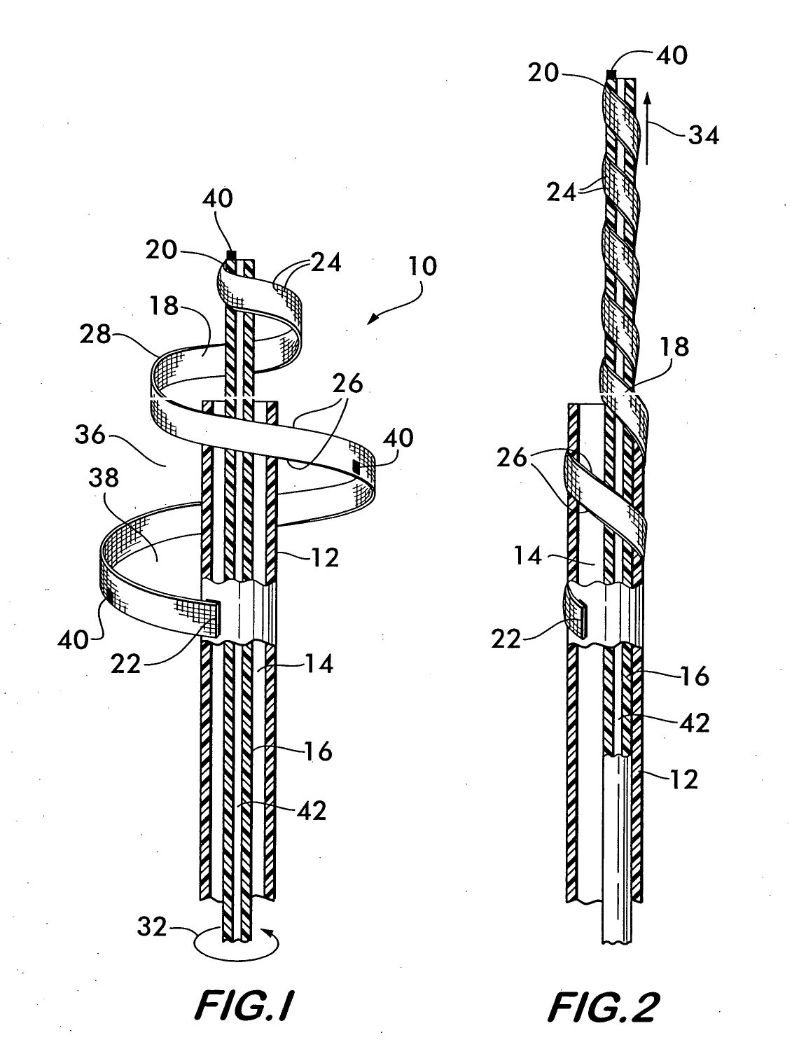

Strip 18 is expandable and contractible about rod 16 and tube 12. FIG. 1 shows strip 18 in its expanded state extending radially outwardly, and FIG. 2 shows strip 18 in its contracted state, drawn inwardly, substantially in proximity to the rod 16 and tube 12. Transition from the expanded to the contracted states is effected by twisting rod 16 relatively to tube 12 in a counterclockwise sense as indicated by arrow 32. Extending the rod 16 relative to the tube 12 in the direction indicated by arrow 34 will also effect transition from the expanded to the contracted states. Preferably, a combination of both twisting and sliding motion is used for greatest control. Opposite motions of the rod 16 and tube 12 are applied to transition from the expanded state to the contracted state. FIGS. 3 and 4 illustrate both the expanded and contracted states for the closed surface embodiment. Transition between the configurations is again effected by relative sliding and twisting motion of rod 16 and tube 12.

When in the expanded state as shown in FIGS. 1 and 3, the strip 18 defines a basket 36 having an opening 38 facing away from the first end 20 of the strip. The opening 38 and basket 36 are adapted to receive an embolus as described below.

Preferably, filamentary members 24 are formed from bio-compatible metal alloys that have a high elastic modulus and a high yield strength. These characteristics allow the filaments to be resilient, flexible and biasable into the compound curves of the helical shape of strip 18. Materials such as stainless steel, nitinol and eligiloy are preferred. It is also feasible to used polymer filaments such as nylon, polyester, polypropylene and polytetrafluoroethylene, either alone or in combination with metal filaments.

In practical embodiments of the snare 10, the strip 18 may be about 1 mm to 3 mm wide, about 0.0008 to 0.002 inches thick and woven from filaments having a diameter between 0.0004 to 0.001 inches. The density of the weave may range between 20 and 1000 filaments per inch, with a preferred density of about 200 filaments per inch to provide sufficient porosity to permit substantial blood flow through the strip when in the open configuration. Porosity is of greater concern for the closed surface embodiment (30 in FIG. 3) than the open helix 28 shown in FIG. 1.

Because snare 10 is intended to be used within arteries of the vascular system, it is advantageous to position radiopaque markers on it so that its position is visible under fluoroscopic devices. For example, tantalum markers 40 may be positioned at the tip of rod 16 and at extreme points of the strip 18 enabling the user to readily determine the snare's position and configuration (i.e., expanded or contracted). Radiopaque filaments may also be interlaced with the filamentary members 24 comprising the strip 18 to enhance visibility.

It may also be advantageous to permit fluids such as contrast dye or medicaments to be injected into the artery during a procedure. To this end, the rod 16 has a duct 42 extending along its length. The duct is in fluid communication with the artery and will permit fluid injection into the blood stream as described below.

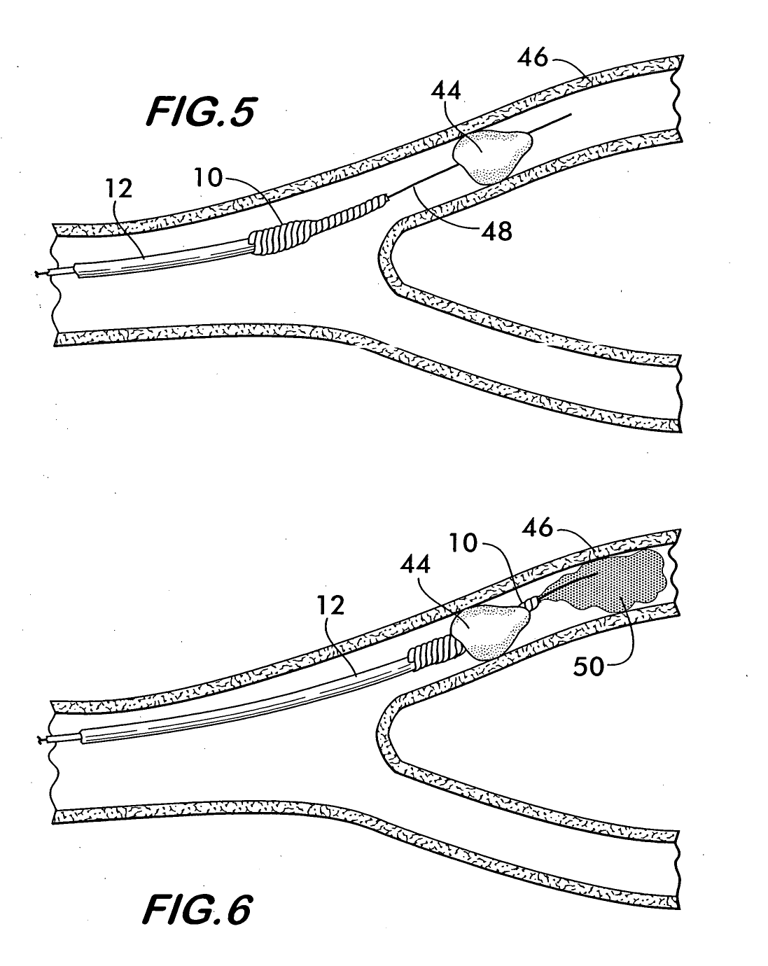

Use or the snare 10 to remove an embolus from an artery is illustrated with reference to FIGS. 5-8. As shown in FIG. 5, an embolus 44 is lodged within an artery 46. A guide wire 48 is positioned within the artery extending past the embolus 44. Snare 10 is guided along the guide wire 48 to the embolus, the guide wire being received within duct 42 of the rod 16.

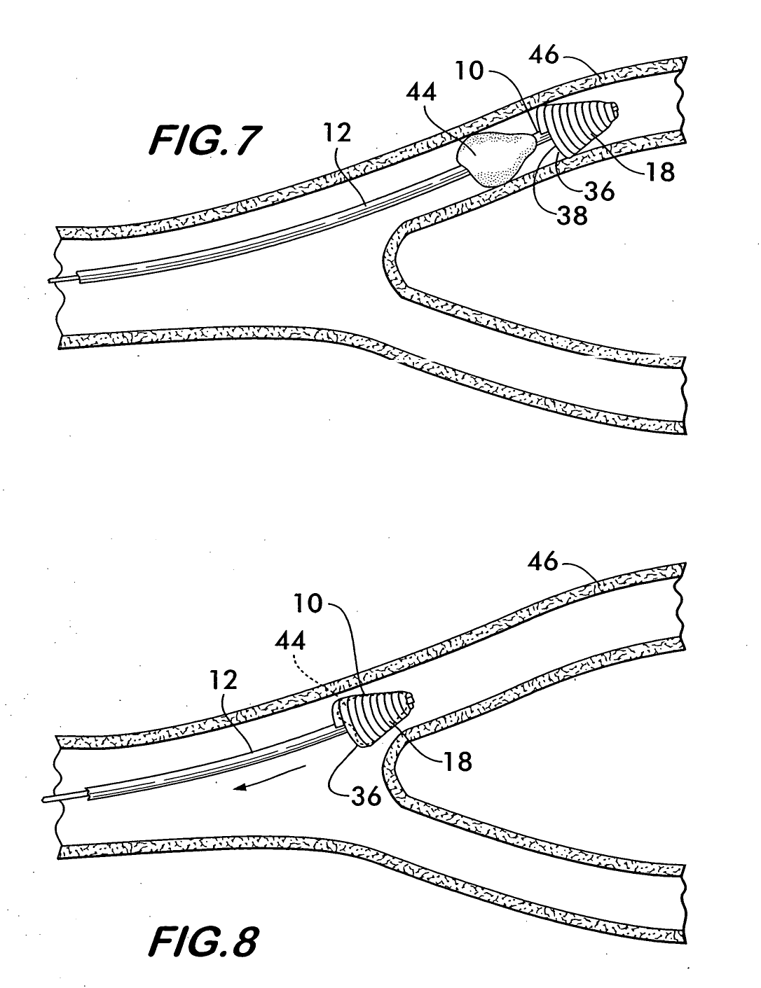

As shown in FIGS. 6 and 7, the snare 10 is pushed past the embolus 44, either through it or between it and the artery wall. Confirmation that the snare is past the embolus may be had by injection contrast dye 50 through duct 42 and seeing it enter the artery downstream of the embolus 44.

As shown in FIG. 7, once the snare 10 is pushed past the embolus it is opened into the expanded state by relative motion of the tube 12 and the rod 16 as described above. The opening 38 faces the embolus 44 and the snare 10 is drawn toward it, capturing the embolus within basket 36. The snare 10 is then closed into the contracted state as shown in FIG. 8 to secure the embolus 44 within the chamber 36. Both the snare and the embolus are removed from the artery 46.

The force applied to expand and contract the snare 10 is largely governed by the elastic and stiffness properties of the materials comprising the strip 18, the biasing force developed within the filamentary members 24, and the relative motion between the rod 16 and the tube 12. It is desirable to control the outward radial force exerted by the strip 18 on the artery 46 as it expands so as not to distend the arterial tissue. Similarly, it is also advantageous to have inward radial force available to securely capture the embolus 44. These radial forces are adjusted by choice of material for the filamentary members as well as their geometric properties such as cross-sectional area and area moment of inertia which affect stiffness, the shape in which the strip 18 is biased, and the technique of manipulating the rod 16 and tube 12 to effect expansion and contraction of the strip 18.

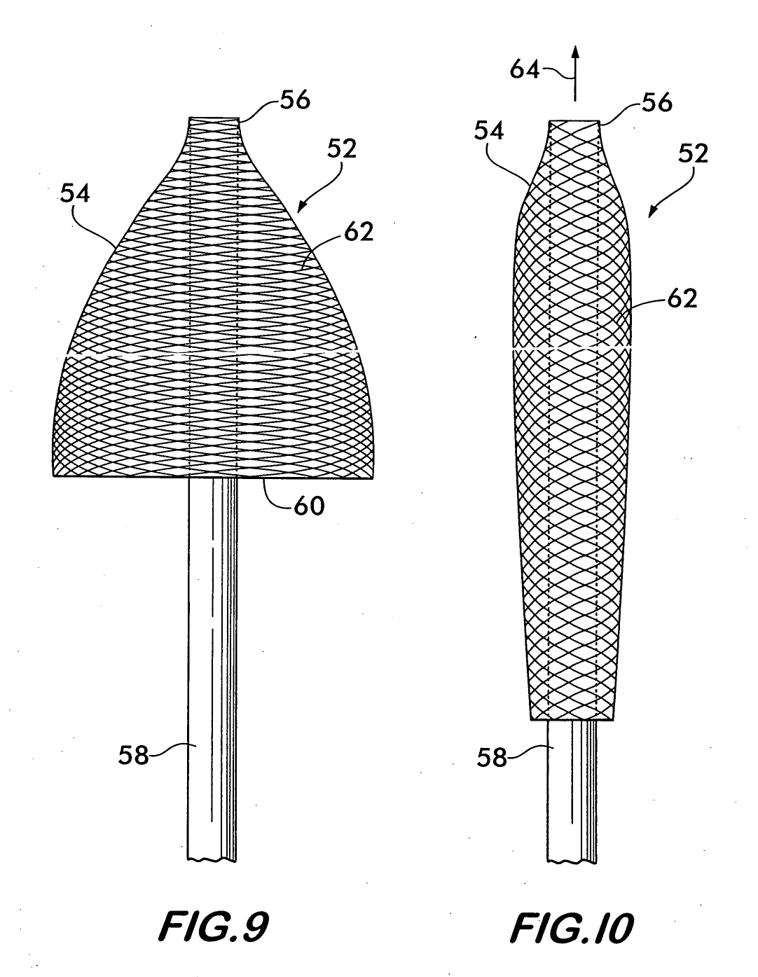

FIGS. 9 and 10 illustrate an alternate embodiment of a snare 52 according to the invention. Snare 52 comprises a flexible sheet that forms a basket 54. Basket 54 is preferably cone-shaped and has an apex 56 at one end that is attached to a flexible, elongated rod 58. Basket 54 also has an opening 60 positioned opposite to the apex 56. The opening 60 provides access to the interior of basket 54 for receiving an embolus.

Preferably, basket 54 is braided of filamentary members 62 that are resiliently biased to nominally assume the expanded state shown in FIG. 9 in the absence of external constraints. Due to the great flexibility of braided structures, the basket 54 may be readily deformed into a contracted state as shown in FIG. 10. When compressed radially, the basket 54 elongates in response, and when expanded radially, the basket shortens. This phenomenon, known as the “trellis effect” allows the snare 52 to be inserted into an artery and deform radially to pass by an embolus in the direction indicated by arrow 64 without damage to the artery wall. Once the basket is past the embolus, the direction of motion is reversed and the basket expands under resilient biasing force to receive the embolus through opening 60. Basket 54 is braided with sufficient longitudinal stiffness to resist column buckling. The embolus may then be removed with the snare 52.

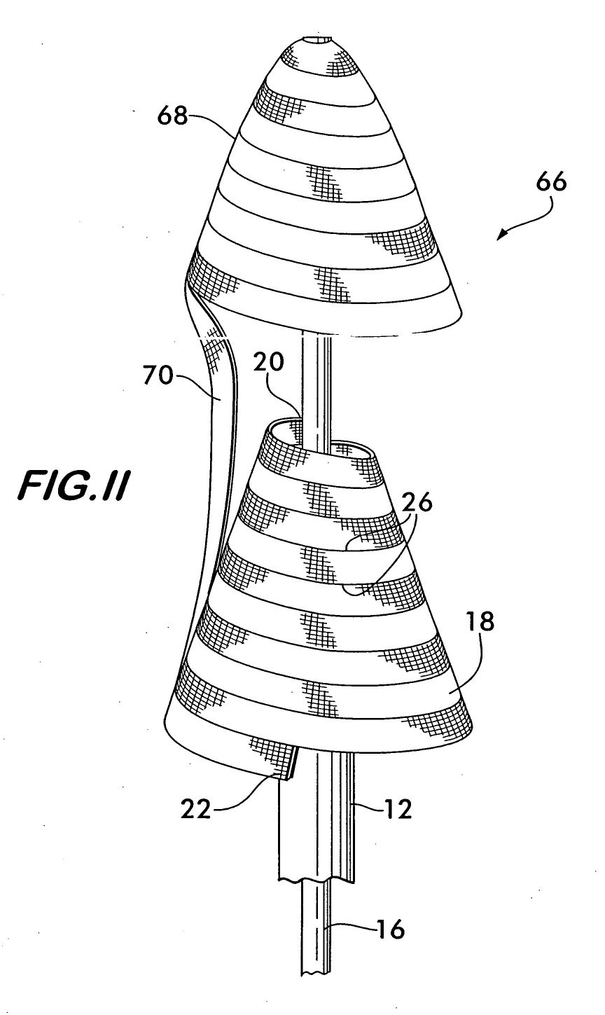

FIG. 11 illustrates another embodiment of a snare 66 according to the invention. Snare 66 is similar in construction and operation to the embodiment 10 of FIGS. 3 and 4, but also includes a second basket 68 formed from a second flexible, resilient sheet positioned downstream of the first basket 36. The second basket 68 is intended to catch any debris that may break free when an embolus is captured by the first basket. Similar to embodiment 52, the second basket may be biased into its expanded configuration and not actively collapsible into its contracted configuration, or, like snare embodiment 10, it may be expandable and collapsible by manipulation of the rod 16 and tube 12. For example, the second basket 68 may be attached to the first basket 36 by a link 70, so that whatever the first basket does is mirrored by the second basket.

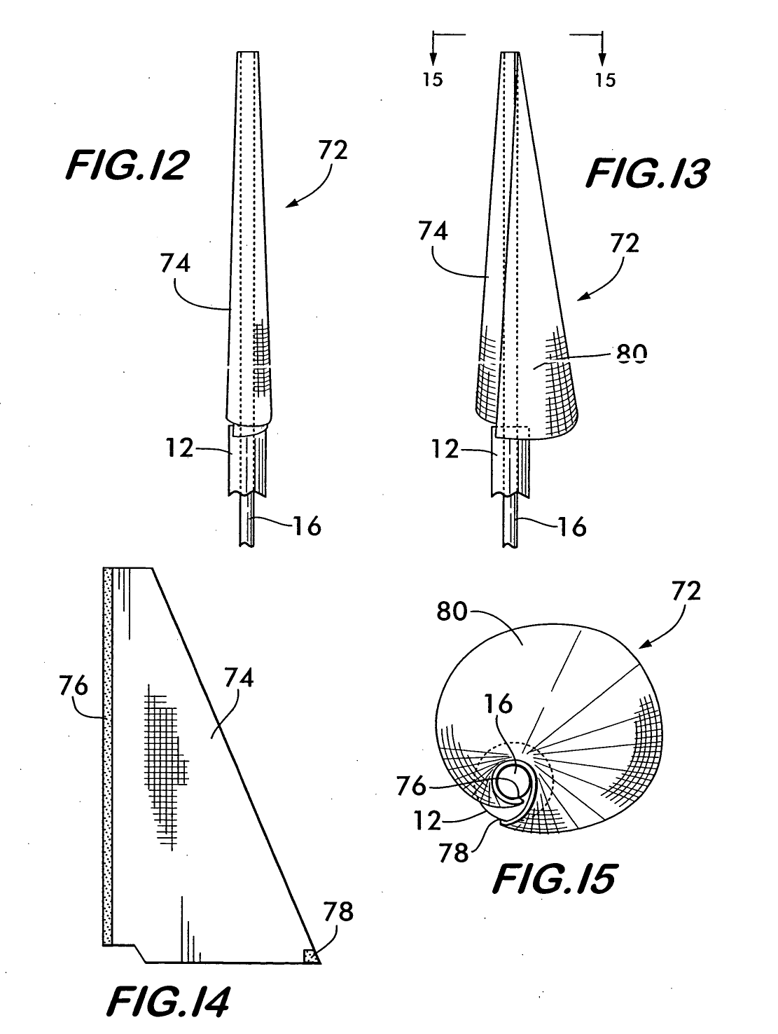

FIGS. 12, 13 and 15 illustrate another embodiment of a snare 72 having a tube 12 within which a rod 16 is movably positioned. A flexible resilient sheet 74, shown in detail in FIG. 14, is attached to both the rod and the tube. The sheet is deformable between a contracted state, shown in FIG. 12, and an expanded state, FIG. 13. Sheet 74 is preferably comprised of interlaced filamentary members made of nylon, polyester, polypropylene, or metals such as stainless steel, nitinol and eligiloy. The sheet may also be a continuous membrane made, for example, from expanded polytetrafluoroethylene.

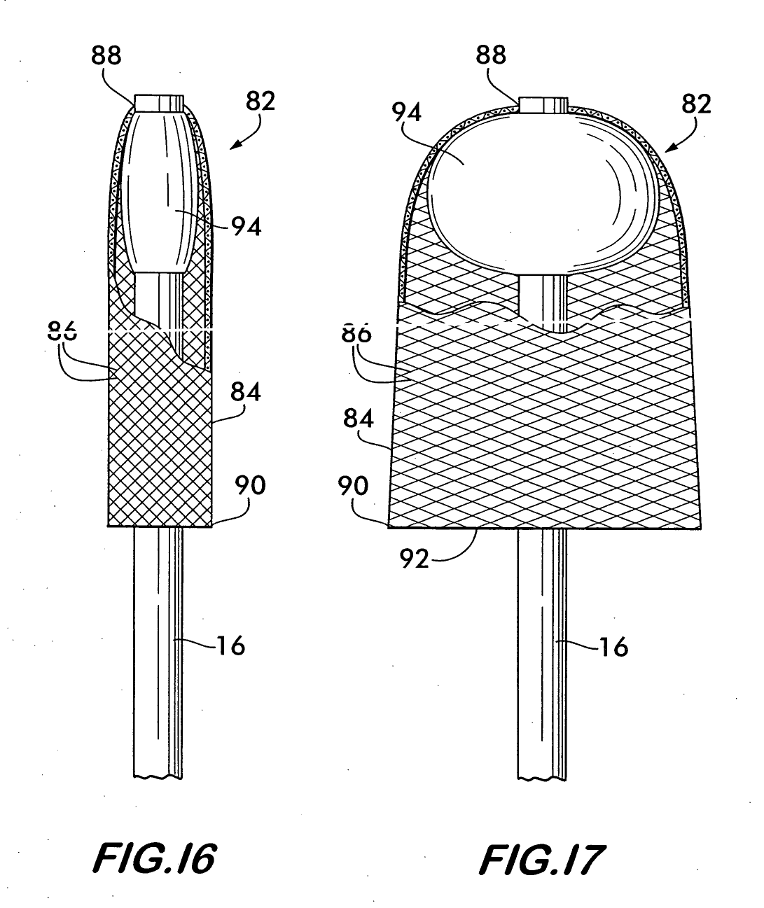

As shown in FIG. 14, sheet 74 is preferably trapezedial in shape with its altitude about three times the length of its base. An elongated edge 76 of the sheet is attached to the rod 16 and a second region 78, positioned at a corner opposite to edge 76, is attached to the tube 12 (see also FIG. 15). This attachment configuration allows the sheet to be expanded into a conical basket 80 upon relative twisting of the tube 12 and the rod 16 in a first direction, as shown in FIGS. 13 and 15. Twisting of the rod and tube in an opposite direction winds the substrate 74 about the rod 16 and into the contracted state shown in FIG. 12.

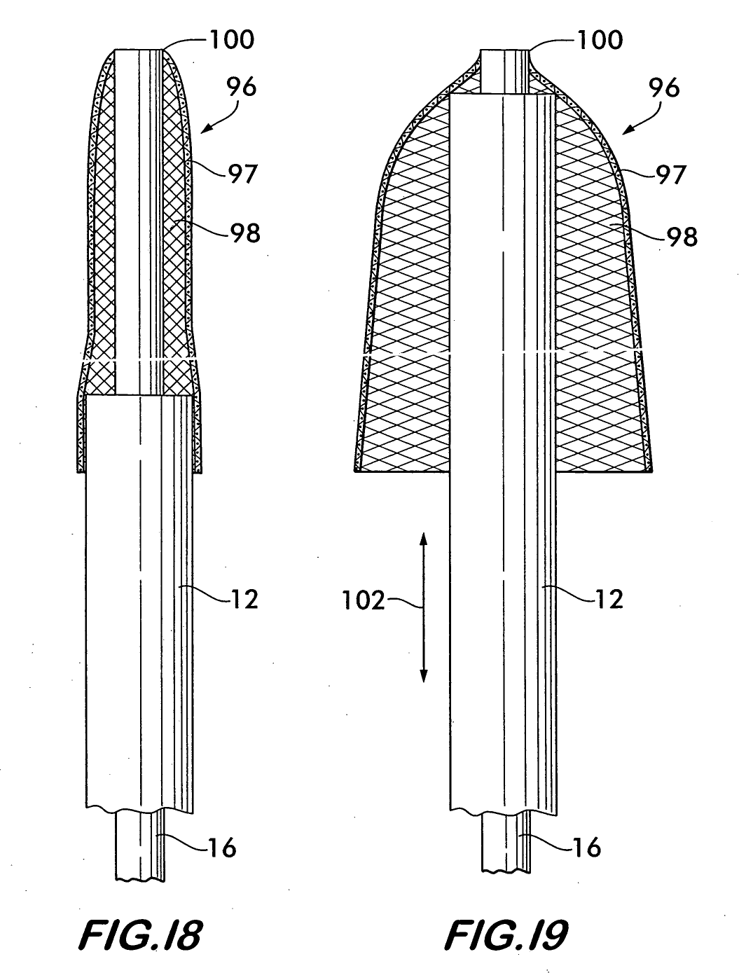

FIGS. 16 and 17 show another snare embodiment 82. Snare 82 comprises a basket 84, formed of a flexible, resilient sheet 86. Sheet 86 is preferably formed of braided filamentary members comprising bio-compatible polymers or metal as described above, although a continuous membrane is also feasible. Sheet 86 has a first end 88 attached to a flexible rod 16, the opposite end 90 forming an opening 92 for receiving an embolus. The sheet 86 is biased so that the basket nominally assumes a contracted state wherein the sheet is adjacent to the rod as shown in FIG. 16. Basket 84 is deformable into an expanded state shown in FIG. 17 by inflating a balloon 94 mounted on rod 16 within the basket 84 and preferably near the first end 88 of sheet 86. Due to the resilient biasing of the sheet, the basket 84 will assume its contracted configuration when the balloon 94 is deflated. Biasing the basket into the contracted configuration as opposed to the expanded configuration provides for a gripping of the embolus by the basket without the need to deform the basket by other means and thus provides a secure engagement as the embolus is withdrawn with the snare 82. The basket is in no danger of inadvertently opening and releasing the embolus, as positive and deliberate steps must be taken to effect embolus release.

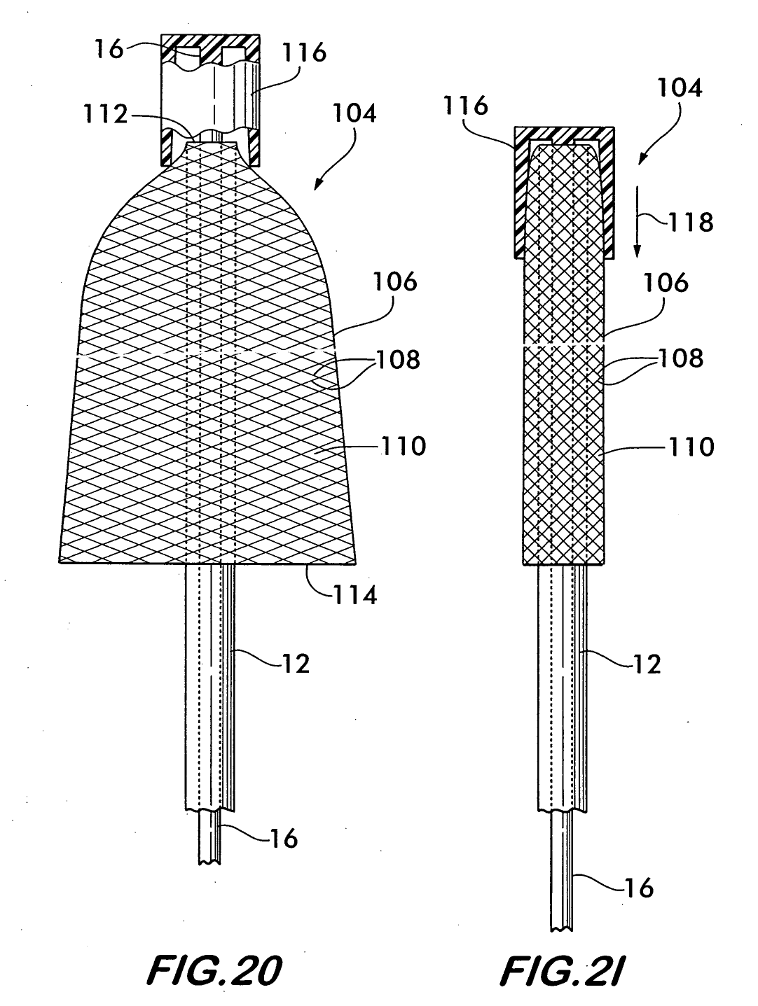

Another snare embodiment 96 is shown in FIGS. 18 and 19. Again, snare 96 comprises a flexible sheet 97 that forms a basket 98 for receiving the embolus. Basket 98 is attached at one end 100 to a flexible elongated rod 16, the opposite end being free and defining an opening. A tube 12 surrounds the rod 16 and is slidable relative to the rod in the directions indicated by arrow 102. Basket 98 is biased so that it assumes a contracted state wherein the sheet 97 is substantially adjacent to rod 16 as shown in FIG. 18. The basket is deformable between the contracted state and an expanded state shown in FIG. 19 by sliding the tube 12 over rod 16 and into the basket 98 so as to engage an inner surface of the basket near the end 100 attached to the rod 16. Engagement of the tube 12 against the inner surface will force the basket to expand radially so that it may receive an embolus. Once the embolus is within the basket the tube 12 may be moved away from end 100 to allow the basket 98 to collapse toward its nominal contracted shape and grip the embolus.

Another embodiment of a snare 104 is shown in FIGS. 20 and 21. A flexible sheet 106, preferably comprised of braided filamentary members 108 forms a basket 110 having one end 112 attached to an elongated flexible tube 12 within which a rod 16 is movably positioned. The opposite end of the basket 110 forms an opening 114 for receiving the embolus. A hollow cap 116 is attached to the end of the rod 16 and extends beyond the basket 110. The sheet is biased so that basket 110 nominally assumes an expanded configuration extending outwardly from tube 12 shown in FIG. 20. Sheet 106 is flexible and resilient, enabling the basket to be deformed into a contracted configuration wherein the sheet is substantially adjacent to the tube 12 as shown in FIG. 21. Deforming the basket from the expanded to the contracted configuration is effected by sliding rod 16 relatively to tube 12 in the direction indicated by arrow 118. This draws the cap 116 over basket 110. The cap has an inner diameter adapted to allow it to pass over but engage the basket. Engagement of the cap with the basket forces the basket to collapse radially into the contracted configuration of FIG. 21. When cap 116 is moved in the opposite direction out of engagement with basket 110, the basket expands back into its nominal open configuration due to the resilient biasing of sheet 106.

Snares according to the invention provide the ability to capture and remove emboli from vascular vessels with significant reliability and convenience and help avoid complications associated with this procedure.

1. A snare for capturing and removing an embolus from a vessel, said snare comprising:
an elongated flexible tube having a bore therethrough;
an elongated flexible rod positioned within said bore, an end of said rod projecting outwardly from said tube, said rod and said tube being movable relatively to one another; and
a flexible sheet having a first portion attached to said rod and a second portion positioned in spaced apart relation to said first portion, said second portion being attached to said tube, said sheet being deformable between a contracted state wherein said sheet is substantially positioned in proximity to said tube, to an expanded state wherein said sheet extends outwardly from said tube to form a basket, said basket having an opening for receiving said embolus, relative motion between said rod and said tube deforming said sheet between said expanded and contracted states.

2. A snare according to claim 1, wherein said flexible sheet comprises an elongated strip having one end attached to said end of said rod and an opposite end attached to said tube, said strip being helically wrapped around said rod and said tube.

3. A snare according to claim 2, wherein said strip is formed of interlaced filamentary members.

4. A snare according to claim 2, wherein said strip has opposite edges extending lengthwise therealong, said strip being biased so as to bring said edges substantially into contact with one another forming a substantially closed surface defining said basket when said strip is in said expanded state.

5. A snare according to claim 1, further comprising a second flexible sheet attached to said rod and positioned in spaced relation to said first named flexible sheet, said second flexible sheet being deformable outwardly from said rod to form a basket having an opening for receiving said embolus.

6. A snare according to claim 5, further comprising a link element extending between said first named and said second sheets, said link element connecting said sheets to one another so that said second sheet is deformed into said expanded state in response to motion of said first named sheet being deformed into said expanded state.

7. A snare according to claim 5, wherein said second flexible sheet is formed of interlaced filamentary members.

8. A snare according to claim 1, wherein said first portion of said flexible sheet comprises an elongated edge, said sheet being attached to said rod along said edge, said second portion of said sheet comprising a corner of said sheet positioned opposite to said edge, said corner being attached to said tube, relative rotational motion between said rod and said tube deforming said sheet between said expanded and contracted states.

9. A snare according to claim 8, wherein said flexible sheet has a trapezoidal shape.

10. A snare according to claim 8, wherein said flexible sheet is formed of interlaced filamentary members.

11. A snare according to claim 1, wherein said rod has a duct therethrough, said duct being adapted for the injection of fluids into said vessel.

12. A snare for capturing and removing an embolus from a vessel, said snare comprising:
an elongated flexible tube having a bore therethrough;
an elongated flexible rod positioned within said bore, an end of said rod projecting outwardly from said tube, said rod and said tube being slidably movable relatively to one another; and
a flexible sheet forming a basket for receiving said embolus, said sheet having a first end attached to said rod and a second end positioned opposite said first end and forming an opening of said basket, said basket being nominally biased into a contracted state wherein said sheet is positioned substantially adjacent to said rod, said basket being deformable from said contracted state to an expanded state wherein said sheet extends outwardly from said rod, relative sliding motion between said rod and said tube causing said tube to engage an inner surface of said basket thereby deforming said sheet outwardly into said expanded state.

13. A snare according to claim 12, wherein said sheet is formed of interlaced filamentary members.

14. A snare according to claim 13, wherein said filamentary members are interlaced by braiding.

15. A snare according to claim 12, wherein said sheet is attached to said end of said rod.

16. A snare according to claim 12, wherein said rod has a duct therethrough, said duct being adapted for the injection of fluids into said vessel.

17. A snare for capturing and removing an embolus from a vessel, said snare comprising:
an elongated flexible tube having a bore therethrough;
an elongated flexible rod positioned within said bore, an end of said rod projecting outwardly from said tube, said rod and said tube being slidably movable relatively to one another;
a cap positioned on said end of said rod, said cap defining a socket with an opening facing said tube, said socket being sized to receive an end of said tube upon relative sliding motion between said rod and said tube; and
a flexible sheet forming a basket for receiving said embolus, one end of said basket being attached to said end of said tube, an opposite end forming an opening, said basket being resiliently biased into an expanded state wherein said sheet extends outwardly from said tube, said basket being deformable from said expanded state into a contracted state wherein said sheet is positioned substantially adjacent to said tube upon relative sliding motion between said rod and said tube inserting said end of said tube within said socket of said cap, said cap engaging and deforming said basket into said contracted state.

18. A snare according to claim 17, wherein said sheet is formed of interlaced filamentary members.

19. A snare according to claim 18, wherein said filamentary members are interlaced by braiding.

20. A snare according to claim 17, wherein said rod has a duct therethrough, said duct being adapted for the injection of fluids into said vessel.

21. A snare for capturing and removing an embolus from a vessel, said snare comprising:
an elongated flexible rod; and
a flexible sheet forming a basket for receiving said embolus, one end of said basket being attached to said rod, the opposite end forming an opening, said basket being biased into an expanded state extending outwardly from said rod, said basket being deformable into a contracted state wherein said sheet is positioned substantially adjacent to said rod upon insertion of said rod into said vessel, said basket resiliently assuming said expanded state upon motion of said rod in a direction removing said cone from said vessel.

22. A snare according to claim 21, wherein said sheet is formed of interlaced filamentary members.

23. A snare according to claim 22, wherein said filamentary members are interlaced by braiding.

24. A snare according to claim 21, wherein said rod has a duct therethrough, said duct being adapted for the injection of fluids into said vessel.

25. A snare for capturing and removing an embolus from a vessel, said snare comprising:
an elongated flexible rod;
a flexible sheet forming a basket for receiving said embolus, one end of said basket being attached to said rod, the opposite end of said basket defining an opening, said basket being biased into a contracted state wherein said sheet is positioned substantially adjacent to said rod; and
a balloon mounted on said rod within said basket, said balloon being inflatable to deform said basket from said contracted state to an expanded state wherein said basket extends outwardly from said rod, said basket resiliently assuming said contracted state upon deflation of said balloon.

26. A snare according to claim 25, wherein said sheet is formed of interlaced filamentary members.

27. A snare according to claim 26, wherein said filamentary members are interlaced by braiding.

28. A snare according to claim 25, wherein said rod has a duct therethrough, said duct being adapted for the injection of fluids into said vessel.
